(19) United States
(12) Patent Application Publication
(10) Pub. No.: US 2014/0275765 A1
(43) Pub. Date: Sep. 18, 2014

(54) PROBE ASSEMBLY AND DISPOSABLE COVER PARTICULARLY FOR USE IN ENDOSCOPE APPLICATIONS OF LOW COHERENCE INTERFEROMETRY

(71) Applicants: Steven C. Gebhart, Durham, NC (US); Norman R. Akens, Lake In The Hills, IL (US); Andrew Black, Johnsburg, IL (US); Andrew Corson, Apex, NC (US); Christopher Draper, Allendale, NJ (US); Perry A. Genova, Chapel Hill, NC (US); Robert Christopher Hall, Apex, NC (US); Tanner Hargens, Charlotte, NC (US); Victor Kopp, Fair Lawn, NJ (US); Matt Penny, Holly Springs, NC (US); Jonathan Singer, New Hope, PA (US); Brian Walsh, Charlotte, NC (US)

(72) Inventors: Steven C. Gebhart, Durham, NC (US); Norman R. Akens, Lake In The Hills, IL (US); Andrew Black, Johnsburg, IL (US); Andrew Corson, Apex, NC (US); Christopher Draper, Allendale, NJ (US); Perry A. Genova, Chapel Hill, NC (US); Robert Christopher Hall, Apex, NC (US); Tanner Hargens, Charlotte, NC (US); Victor Kopp, Fair Lawn, NJ (US); Matt Penny, Holly Springs, NC (US); Jonathan Singer, New Hope, PA (US); Brian Walsh, Charlotte, NC (US)

(21) Appl. No.: 14/213,138

(22) Filed: Mar. 14, 2014

Related U.S. Application Data
(60) Provisional application No. 61/788,784, filed on Mar. 15, 2013.

(51) Int. Cl. A61B 1/00; A61B 1/07

(52) U.S. Cl. CPC A61B 1/00059; A61B 1/07; A61B 1/00142
USPC 600/103; 600/182; 600/121; 600/125

(57) ABSTRACT

A fiber optic probe assembly comprises an optical engine and a fiber optic probe. A proximal end connector comprises a self-aligning connector for connecting optical fibers of the fiber optic probe to the optical engine and an integrated fiber optic-based polarizer. A disposable cover is provided for enclosing at least a portion of a fiber optic probe assembly. The disposable cover comprises an elongated tubular housing and a sheath portion adapted to receive at least a portion of the fiber optic probe between ends of the housing. A handle is disposed on a proximal end of the housing, the handle comprising a self-tensioning device. An optical window is disposed on a distal end of the housing, the optical window adapted to provide for alignment with optical elements of the fiber optic probe.

### CROSS-REFERENCES

This application is related to U.S. provisional application No. 61/788,784, filed Mar. 15, 2013, entitled “PROBE ASSEMBLY AND DISPOSABLE COVER PARTICULARLY FOR USE IN ENDOSCOPE APPLICATIONS OF LOW COHERENCE INTERFEROMETRY”, naming Robert Christopher Hall as the inventor. The contents of the provisional application are incorporated herein by reference in their entirety, and the benefit of the filing date of the provisional application is hereby claimed for all purposes that are legally served by such claim for the benefit of the filing date.

### BACKGROUND

A fiber optic probe assembly is described and, more particularly, a fiber optic probe assembly for use in endoscopic applications of low coherence interferometry, including Fourier domain, angle-resolved low coherence interferometry.

The fiber optic probe assembly includes a proximal end connector that provides a self-aligning connection of the optical fibers of the probe to the optical engine of the low coherence interferometry device that allows the interchange or replacement of fiber optic probes without laborious re-alignment of the optical pathways. The invention also relates to a disposable cover that includes a self-tensioning device on the proximal end of the cover and an optical window on the distal end that provides for alignment with the optical elements of the distal end of the fiber optic probe.

Systems including Low-Coherence Interferometry (LCI) have been used to examine tissue surfaces and/or structural features of cells in tissue. LCI typically utilizes a broadband light source with low temporal coherence. Light from the low coherence source is split by a beamsplitter into a reference beam and an input beam to a sample. An inteferometer is configured such that the reference beam is combined with an optical field returning from the sample, producing an interference pattern between the reference beam and the returning sample beam. Interference is achieved when path length delays of the interferometer are matched with the coherence time of the light source. Axial resolution of the system is determined by the coherence length of the light source and is typically in the micrometer range, suitable for the examination of tissue samples. Several LCI devices have been developed for clinical use with endoscopy. In particular, Fourier domain, angled-resolved LCI (fa/LCI) has been described for in vivo examination of tissue at rapid rates.

Previous fa/LCI systems designed for endoscopic applications have had integrated fiber optic probes that required a trained engineer to dismantle the system to swap or replace the probe. Furthermore, installation of a new fiber optic probe required internal hardware adjustment of the system and testing using a known standard to verify correct installation. What is needed is a fiber optic probe that can be replaced or is interchangeable with minimal adjustment. Ideally, the new fiber optic probe can be properly installed by users in the field, for use in endoscopic applications.

### SUMMARY OF THE INVENTION

A fiber optic probe assembly comprises a fiber optic probe comprising optical fibers for delivery of emitted light and collection of remitted light, a connector at a proximal end of the fiber optic probe that provides a self-aligning connection of the optical fibers of the fiber optic probe to the optical engine of a low coherence interferometry device, and an optical element affixed to and aligned with the optical path of a distal end of the fiber optic probe. The fiber optic probe assembly allows the replacement or interchange of fiber optic probes connected to a low coherence interferometry device without laborious re-alignment of the optical pathways.

A proximal end connector is provided for a fiber optic probe assembly. The proximal end connector comprises an integrated fiber optic-based polarizer. The proximal end connector also optionally comprises embedded memory and optional RFID tag adapted to electronically track and control attachment and removal of the fiber optic probe assembly via software for calibration purposes. In addition, the embedded memory may also contain a unique identifier for the fiber optic probe assembly as well as detection of connection and disconnection along with an event counter incorporated into every fiber optic probe assembly.

A disposable cover is also provided for a fiber optic probe assembly. The disposable cover comprises a handle comprising a self-tensioning device at a proximal end of the disposable cover and an optical window at a distal end of the disposable cover, the optical window providing for alignment with the optical elements of a distal end of the fiber optic probe. In one embodiment, the self-tensioning device comprises a spring disposed inside the handle at the proximal end of the disposable cover, wherein the handle is coupled to the proximal end of the fiber optic probe assembly. In another embodiment, the distal end of the disposable cover comprises an optical window adapted to align and associate with an optical element aligned and associated with the distal end of the fiber optic probe.

It is another object of the invention to provide a low coherence interferometry system comprising a fa/LCI device, a fiber optic probe assembly configured to allow replacement or interchange of a fiber optic probe, and a disposable cover having a proximal end comprising a self-tensioning handle capable of being coupled to the fiber probe assembly and a distal end comprising an optical window that aligns and associates with an optical element aligned and associated with the fiber optic probe.

### BRIEF DESCRIPTION OF THE DRAWINGS

For a more complete understanding of the present invention, reference should now be had to the embodiments shown in the accompanying drawings and described below. In the drawings:

FIG. 1 is a schematic view of an exemplary endoscope.

FIG. 2 is a schematic view of a fiber optic probe for an exemplary fa/LCI system employed in an instrument channel of an endoscope for examining tissue in vivo.

FIG. 3A is a schematic view of an exemplary embodiment of a fiber-optic fa/LCI system.

FIG. 3B is a schematic view of sample illumination and scattered light collection with a distal end of a fiber optic probe in an fa/LCI system in FIG. 3A.

FIG. 3C is an image of the illuminated distal end of the fiber optic probe of an fa/LCI system as shown in FIG. 3A.

FIG. 4 is a side elevation view of a proximal end connector for a fiber optic probe for use in an embodiment of a fiber optic probe assembly.

FIG. 5 is a top plan view of the proximal end connector as shown in FIG. 4.

FIG. 6 is a top plan view of the proximal end connector as shown in FIG. 5 with a top housing removed.

FIG. 7 is a longitudinal cross-section view of a fiber optic probe tip for use in an embodiment of a fiber optic probe assembly.

FIG. 8 is a bottom plan view of the proximal end connector as shown in FIG. 5 with the bottom housing removed.

FIG. 9 is an opposite side elevation view of the proximal end connector as shown in FIG. 4.

FIG. 10 is a perspective view of an optical port connector for an optical engine that would mate with the proximal end connector as shown in FIG. 4.

FIG. 11 is a flow diagram showing the light paths for an embodiment of a fiber optic probe assembly comprising a proximal end connector and a fiber optic probe with probe tip connected to an exemplary optical engine for low coherence interferometry.

FIGS. 12 and 13 are exploded perspective views of one embodiment of a disposable cover and a distal tip of the disposable cover, respectively.

FIGS. 14 and 15 provide an exploded view and an assembled view of a disposable cover showing an alternative embodiment of a disposable cover.

FIG. 16 provides an isometric view of one embodiment of a disposable cover 200.

FIGS. 17 and 18 provide a profile view of one embodiment of a disposable cover 200 and a section view of the distal end of the disposable cover.

FIGS. 19 and 20 provide a profile view of one embodiment of a disposable cover 200 and a section view of the proximal end of the disposable cover for the first embodiment.

FIG. 21 provides a top view of the proximal end of one embodiment of a disposable cover inserted on a fiber optic probe 100.

FIG. 22 provides a top view of the proximal end of one embodiment of a disposable cover inserted on a fiber optic probe in a locked position.

FIGS. 23A-I show embodiments of distal tip of a fiber optic probe comprising an optical housing, a GRIN lens, optical window, disposable sheath, fiber bundle for transmitting remitted light from the tissue sample, PM fiber for delivery of emitted light from optical engine to tissue sample.

FIG. 23A (upper) shows an exploded view of the components on one embodiment, and a fully configured fiber optic probe tip.

FIG. 23B shows an exploded view of an optical housing for the optical element and a distal ferrule that receives the optical housing, where the distal ferrule is attached to the disposable sheath configured such that the optical path of the optical fibers in the fiber optic probe are aligned with the optical element.

FIGS. 23C and 23D show optical housings for the GRIN lens optical element aligned with the optical path of the optical fibers in the fiber optic probe.

FIG. 23E shows a distal probe tip arrangement where the disposable cover is crimped to hold it to the optical fiber bundle.

FIG. 23F shows an alternate embodiment where a proximal ferrule is used to position the optical fiber bundle for alignment of the optical path.

FIG. 23G shows an alternate embodiment of the optical housing having a thermoformed cover with a compliant spring feature to position the optical fiber bundle and affix the probe tip to the optical fiber bundle.

FIG. 23H shows three views of a disposable optical housing for an optical element that can be affixed to a disposable sheath cover and aligns with the optical fiber bundle.

FIG. 23I shows three views of an alternate embodiment of the probe tip of FIG. 23H.

FIG. 24 is a perspective view of a proximal end connector with disposable cover handle coupled to the optical port on a box containing an optical engine.

### DETAILED DESCRIPTION

The detailed description of the following embodiments provides the information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

The invention described herein relates to a fiber optic probe assembly for use in endoscopic applications of low coherence interferometry, including Fourier domain, angle-resolved low coherence interferometry. The fiber optic probe assembly comprises a fiber optic probe comprising optical fibers for delivery of emitted light and collection of remitted light, a connector at the proximal end of the fiber optic probe that provides a self-aligning connection of the optical fibers of the fiber optic probe to the optical engine of the low coherence interferometry device, and an optical element affixed to and aligned with the optical path of distal end of the fiber optic probe. The fiber optic probe assembly allows the replacement or interchange of fiber optic probes connected to a low coherence interferometry device without laborious re-alignment of the optical pathways. The invention also relates to a disposable cover for the fiber optic probe assembly that comprises a handle further comprising a self-tensioning device on the proximal end of the cover and an optical window on the distal end of the disposable cover that provides for alignment with the optical elements of the distal end of the fiber optic probe.

The fiber optic probe assembly with a self-tensioning disposable cover capable of being coupled to the fiber optic probe assembly is designed to be used with various systems employing LCI. Such systems are described in U.S. Pat. RE42,497, entitled “Fourier Domain Low-Coherence Interferometry for Light Scattering Spectroscopy Apparatus and Method”, in U.S. Pat. No. 7,595,889 entitled “Systems and Methods for Endoscopic Angle-Resolved Low Coherence Interferometry”, in co-pending U.S. Patent Application Publication No. 2008/0021276 (Ser. No. 11/780,879) entitled “Protective Probe Tip, Particularly for Use on a Fiber-Optic Probe used in an Endoscopic Application”, in co-pending U.S. Patent Application Publication No. 2012/0127475 A1 (Ser. No. 13/305,095) entitled “Apparatuses, Systems, and Methods for Low-Coherence Interferometry”, and in co-pending U.S. Patent Application Publication No. 2009/0177094A1 (Ser. No. 12/350,689), entitled “Systems and Methods for Tissue Diagnostic, Monitoring, and/or Therapy”, each of the above incorporated by reference herein in its entirety.

Embodiments of an LCI-based apparatus, system, and method described above and in this application can be clinically viable means for assessing tissue health without the need for tissue extraction via biopsy or subsequent histopathological evaluation. The embodiments of the LCI-based apparatus, system, and method can be applied for a number of purposes including, but not limited to: early detection and screening for dysplastic tissues, disease staging, monitoring of therapeutic action, and guiding the clinician to biopsy sites. Some potential target tissues include the esophagus, the colon, the stomach, the oral cavity, the lungs, the bladder, and the cervix. The non-invasive, non-ionizing nature of LCI and the optical probe means that it can be applied frequently without adverse effect. The provision of rapid results through the use of the fa/LCI systems and processes disclosed herein greatly enhance its widespread applicability for disease screening.

A typical biopsy endoscope 26 is illustrated in FIG. 1. The endoscope 26 may have a camera, aperture, or other imaging device 28 on a distal end of a shaft 30, which may be rigid or flexible, for visual inspection of tissue. An eyepiece 31 is used to review the images of the tissue captured by the aperture or imaging device 28. High-definition images may also be captured using video techniques. The endoscope 26 may have one or more channels 32 for introducing light, and one or more instrument or accessory channels 34. As an example, a biopsy endoscope may have three channels, an integrated channel for visual inspection, an instrument channel through which biopsy forceps may be passed, and an instrument channel through which a fiber optic probe assembly may be passed. There may also be channels for air and water, and endoscopes may have visual illumination sources at the distal end.

FIG. 2 illustrates an example of a fa/LCI system 40 employing a fiber optic probe assembly 45 in an instrument channel 41 of an endoscope 42 to perform guided or optical biopsy of tissue during a patient procedure or examination, and which may be used in the above-described methods, processes and techniques. This configuration may be useful in that an endoscope enables guided biopsy where the integrated fa/LCI system allows the operator to determine tissue status in vivo and use that information to collect biopsies from the areas of higher concern. As illustrated in FIG. 2, the fa/LCI system 40 is provided and interfaces with a computer 43 to control the operation of and receive data from the fa/LCI system 40 regarding the tissue examined. In this regard, the computer 43 is interfaced with the fa/LCI system 40 via a communication line 44. A fiber optic probe assembly 45 connected to the optical port 49 of the optical engine inside the fa/LCI system 40 is passed down the instrument channel 41 of the endoscope 42 to direct light to the tissue of interest and to collect depth-resolved angular distributions of scattered light from the tissue for evaluation and diagnosis. A second instrument channel 46 can be provided on the endoscope 42 for receiving light, air, water, or other substance via a shaft 47 to assist in the examination of tissue 48. The physician can examine or monitor the tissue using the eyepiece 39 or other imaging system connected to the endoscope 42 as the fa/LCI system 40 scans the tissue 48 of interest in real time. A shaft 51 of the endoscope 42 can be moved within the patient to examine the tissue 48 of interest.

A non-limiting example of the optical engine and associated fiber optic probe of a typical fa/LCI system based on a modified Mach-Zehnder interferometer is illustrated in FIGS. 3A and 3B. Broadband light 50″ from a fiber-coupled superluminescent diode SLD source 52″ (e.g., Superlum, Po=20 mW, λo=830 nm, Δλ=18 nm, coherence length=6.3 μm) is split into a sample arm delivery fiber 56″ and a reference arm delivery fiber 54″ by a 95/5 fiber splitter FS 120′ (e.g., manufactured by AC Photonics). The sample arm delivery fiber 56″ can consist of either of the following, for example: (1) a single mode fiber with polarization control integrated at the tip; or (2) a single mode fiber that is a polarization maintaining fiber. A fiber optic probe is assembled by affixing the delivery fiber 56″along a ferrule 154 at a distal end of a fiber bundle 156 such that the end face of the delivery fiber 56″ is parallel to and flush with the face of the fiber bundle 156. Ball lens L1 155 (e.g., f1=2.2 mm) is positioned one focal length from the face of the fiber optic probe and centered on the fiber bundle 156, offsetting the delivery fiber 56″ from the optical axis of lens L1 155. This configuration, which is also depicted in FIG. 3B, produces a collimated beam 160 (e.g., P=9 mW) with a diameter of 0.5 mm incident on the sample 58″ at an angle of 0.25 radians, for example.

Referring to FIG. 3B, scattered light from the sample (returning arrows) is collected by lens L1 155 and, via the Fourier transform property of the lens L1 155, the angular distribution of the scattered field is converted into a spatial distribution at the distal face of the multimode coherent fiber bundle 156 (e.g., Schott North America, Inc., length=840 mm, pixel size=8.2 μm, pixel count=13.5K) which is located at the Fourier image plane of lens L1 155. The relationship between vertical position on the fiber bundle, y′, and scattering angle, θ, is given by y′=f₁θ. As an illustration, the optical path of light scattered at three selected scattering angles (arrows) is shown in FIG. 3B. Overall, the angular distribution is sampled by approximately 170 individual fibers, for example, across a vertical strip of the fiber bundle 156″, as depicted by the highlighted area in FIG. 3C. The 0.2 mm, for example, thick ferrule (d1) separating the delivery fiber 56″ and fiber bundle 156 limits the minimum theoretical collection angle (θ_{min,th}=d₁/f₁) to 0.09 radians in this example. The maximum theoretical collection angle is determined by d1 and d2, the diameter of the fiber bundle, by θ_{max,th}=+(d₁+d₂)/f₁ to be 0.50 radians. Experiments using a standard scattering sample 162 indicate the usable angular range to be θₘᵢₙ=0.12 radians to radians d₁, for example, can be minimized by fabricating a channel in a distal ferrule 163 (FIG. 3A) and positioning the delivery fiber 56″ in the channel.

The fiber bundle 156 of the fiber optic probe is spatially coherent, resulting in a reproduction of the collected angular scattering distribution at the proximal face. Additionally, as all fibers in the fiber bundle 156 are path length matched to within the imaging depth of the system, the optical path length traveled by scattered light (returning arrows) at each angle is identical. The angular distribution exiting a proximal end 164 of the fiber bundle 156 is relayed by a 4f imaging system of L2 166 and L3 132 (f₂=3.0 cm, f₃=20.0 cm) to the input slit 88″ of the imaging spectrograph 69″ (e.g., Acton Research, InSpectrum 150). The theoretical magnification of the 4f imaging system is (f₃/f₂) 6.67 in this example. Experimentally, the magnification was measured to be M=7.0 in this example with the discrepancy most likely due to the position of the proximal end 164 of the fiber bundle 156 with relation to lens L2 166. The resulting relationship between vertical position on the spectrograph slit 88″, y, and θ is y=Mf₁(θ−θₘᵢₙ). The optical path length of the reference arm is matched to that of the fundamental mode of the sample arm. Light 167 exiting the reference fiber 54″ is collimated by lens L4 168 (e.g., f=3.5 cm, spot size=8.4 mm) to match the phase front curvature of the sample light and to produce even illumination across the slit 88″ of the imaging spectrograph 69″. A reference field 170 may be attenuated by a neutral density filter 172 and mixed with the angular scattering distribution at beamsplitter BS 174. Mixed fields 176 are dispersed with a high resolution grating (e.g., 1200 lines/mm) and detected using an integrated, cooled CCD (not shown) (e.g., 1024×252, 24 μm×24 μm pixels, 0.1 nm resolution) covering a spectral range of 99 nm centered at 840 nm, for example.

The mixed fields 176, a function of wavelength, λ, and θ, can be related to the signal and reference fields (Es, Er) as:

I(λₘ,θₙ)= [symbol] |Eᵣ(λₘ,θₙ)|² [symbol] + [symbol] |Eₛ(λₘ,θₙ)|² [symbol] +2Re [symbol] Eₛ(λₘ,θₙ)Eᵣ^{*}(λₘ,θₙ)cos(φ) [symbol] , (7)

where φ is the phase difference between the two fields, (m,n) denotes a pixel on the CCD, and [symbol] . . . [symbol] denotes a temporal average. I(λm,θn) is uploaded to a personal computer (PC) and processed in 320 ms to produce a depth and angle-resolved contour plot of scattered intensity. The processing of the angle-resolved scattered field to obtain depth and size information described above can then be used to obtain angle-resolved, depth-resolved information about the sample 58″ using the scattered mixed fields 176 generated by the apparatus in FIG. 3A.

In FIG. 4, there is shown an embodiment of a proximal end connector for a fiber optic probe assembly, generally designated at 100. The proximal end connector 100 of the fiber optic probe assembly is a light conduit for the light emitted from an optical engine to the delivery optical fiber for transmitting light to the tissue sample and for the remitted light returned from the tissue sample back to the optical engine by way of a probe connector 113 shown in FIG. 5. The emitted light enters the fiber optic probe through a modified male Fiber-optic Connector/Physical Contact (FC/PC) connector 107 that is part of the probe connector 113 in FIG. 5. Referring to FIG. 6, a single mode fiber 126 for delivering emitted light from the optical engine and through the probe connector is connected to a fiber polarizer 124 by a fiber splice 125. The output side of the fiber polarizer 124 is connected to a polarization maintaining (PM) optical fiber 122 by another fiber splice (FIG. 7). Polarized light travels through a distal end 121 of the PM optical fiber and is focused through a focusing element 115 (Gradient Index (GRIN) lens) onto the tissue sample. The focusing element is optically adhered to the fiber bundle tip using a UV cured adhesive 116. The remitted light scattered from the tissue sample is collected and focused through the optical element back onto a spatially-coherent multi-fiber bundle 120. The fiber bundle transmits the remitted light back to the proximal end connector 100 of the fiber optic probe assembly and the probe connector 113. The proximal end of the fiber optic bundle is encased in a steel ferrule 127 that is centered in another male FC/PC connector 109 that is part of the proximal end connector.

The fiber optic probe of the fiber optic probe assembly further comprises (FIG. 7) the integrated spatially-coherent fiber optic bundle 120 for transmitting remitted light from the sample, the polarization maintaining (PM) delivery optical fiber 122, a distal probe tip 101 comprising an optical element for delivery of emitted light and collection of remitted light, a strain relief 103 (FIG. 4) for preventing kinking of the fiber optic probe 102, a support housing 104 for mating with a disposable cover, a mating pin 105 for locking the disposable cover, a bottom housing 106 for encasing the bottom half of the connector, the fiber connector 107 for connecting the sample light fiber from the optical engine with the delivery light fiber of the probe, an electrical connector 108 for connecting the electronic memory contained in the proximal end connector to the optical engine, the multi-fiber connector 109 for connecting the spatially-coherent multi-fiber bundle to the optical engine, a top housing 110 for encasing the top half of the connector, a locking nut 111 for connecting the probe to the optical port of the optical engine with a threaded connection, and a D shaped connector housing 112 for aligning the proximal end connector 113 of the fiber optic probe assembly to the mating connector on the optical engine.

Referring to FIG. 7, the probe tip 101 contains a distal housing 114 for protecting the contents of the probe tip, a focusing element 115 for delivering and receiving light, an optical adhesive 116 for adhering the optical components, a housing spacer 117 for filling the gap in the housing, an adhesive 119 for adhering the components and filling gaps, and the distal end of the fiber optic probe 102 for transmitting light through the probe. The fiber optic probe further comprises 102 the multi-fiber bundle 120 for spatially coherent transmission of the remitted light back to the optical engine, the polarization maintaining fiber 122 that is a delivery fiber for transmission of the sample light from the fiber polarizer in the proximal connector to the distal surface 121, a bundle cover 123 for protecting the fibers while being flexible, a distal ferrule 118 for holding the optical fibers on the distal end. Referring to FIG. 6, the proximal end connector 100 comprises a proximal ferrule 127 for holding the fibers on the proximal end, and a proximal sheath 128 for protecting the proximal end of the fiber bundle. These elements are rigidly held in the proximal end connector and mounted to the internal rigid body 129 of the proximal end connector.

Referring to FIGS. 8 and 9, the proximal end connector 100 contains an electronic component 130 for data storage, including counting number of uses, deployment time, identification, authenticity, etc. The electronic component may optionally further comprise a RFID tag or similar electronic identification device. The electronic component is connected to the internal rigid body 129 of the proximal end connector. The electronic component is electrically connected to the electrical connector 108 through a set of wires that are part of the electrical connector. The proximal end connector has a rigid form and may be employed with a variety of displacement angles 131 derived from the shapes of the bottom housing 106 and the top housing 110).

Referring to FIG. 10, an optical port connector 150 contains a body 151 for mounting and a “D”-shaped insert 152 for alignment, a fiber optic coupler 153 for connecting the FC/PC connector 154 of the light source 171 to the fiber connector 107 of the probe, an electrical connector 155 for connecting the electronic connector 108 in a probe to an electrical computer connector 156, a threaded connection 157 for providing a mechanical connection with a locking nut 111 of a probe, a multi-fiber connector 158 for connecting the multi-fiber connector 109 in a probe to an optical alignment tube 159 of an optical engine.

Referring to FIG. 11, the light path starts with a light source 171 in the optical engine. The emitted light is transmitted by single mode optical fiber 172 that is connected through a FC/PC connector 154 and FC/PC coupler to the FC/PC connector 107 of the proximal end connector. The light entering the probe is transmitted by single mode optical fiber that is optionally also a polarization maintaining optical fiber 172 that is fiber spliced 125 to a fiber polarizer 124 present in the proximal end connector. The now polarized light leaves the polarizer which is fiber spliced 125 to polarization maintaining optical fiber 122. The polarized light leaves the polarization maintaining optical fiber and goes through the optical element 115. The light leaves the probe on a set path 173 and is scattered off an object 174 such as a tissue sample. The remitted, scattered light returns to the fiber optic probe on a path 175 and is collimated through the optical element 115. The collimated light passes through the multi-fiber bundle 120 to a separate optical connector 109 in the proximal end connector. The light passes through the proximal end connector aligned to the multi-fiber connector 158 mounted in the optical engine. The light then travels through the free space optics including an interferometer 176 of the optical engine and enters the spectrometer 177 of the optical engine. The division of the optical engine and the probe is represented by the dashed line 178. Only the path of the sample beam is shown in FIG. 11.

The use of a GRIN lens as an optical element in the probe tip 101 provides a flat surface that facilitates a zero air gap between the fiber optic probe tip and the optical window of a disposable cover. A preferred GRIN lens will have a zero working distance that allows the facile placement of the lens in the probe tip. The GRIN lens also acts to collimate the emitted light and remitted light passing through it. In one embodiment, a GRIN lens diameter of 1.8 mm is used to cover the face of the fiber optic bundle and illumination fiber while fitting inside the working channel of the endoscope, and the lens length (4.3 mm) is then a derivative to get the zero working distance.

In another embodiment, the fiber optic probe tip 101 comprises a single machined tube that combines tubes 114 and 117 that includes rigid or flexible features for alignment of the GRIN lens with the fiber optics of the distal end of the fiber optic probe. In a further embodiment, the single machined tube can comprise locking features with a disposable cover or with the distal ferrule 118 of the fiber optic probe bundle 102. These and other similar embodiments are set forth in FIGS. 23A-23I and the accompanying figure legends.

The proximal end connector described herein allows the fiber optic probe assembly to achieve certain technical characteristics when connected to the optical port of the optical engine. First, the proximal end connector provides a means for the quick connection and disconnection of the fiber optics in the fiber optic probe assembly at the optical port of the optical engine including the polarization maintaining (PM) delivery optical fiber, alignment of the fiber optics of the fiber optic probe into the free space optics of the optical engine, and electronic connection of the embedded memory of the proximal end connector into the electronics of the optical engine. Thus, a fiber optic probe assembly is provided that can be interchanged or replaced by users in the field.

The embedded memory and optional RFID tag of the proximal end connector also allows the ability to electronically track and control attachment and removal of the fiber optic probe assembly using computer software for calibration purposes. In addition, the embedded memory may also contain a unique identifier for the fiber optic probe assembly, as well as detection of connection and disconnection along with an event counter incorporated into every fiber optic probe assembly.

The proximal end connector of the fiber probe assembly also provides the repeatable alignment and registration of the fiber optic bundle within the sample arm of the optical engine. Alignment in the X, Y, and Z dimensions is achievable to within +/−50 microns. Angular registration of +/−1 degree is achievable for the repeatable connection and disconnection of the fiber optic probe relative to “twelve o'clock” position of the input tube collimator of the optical engine. In addition, the proximal end connector permits repeatable coupling of the polarization maintaining delivery fiber over multiple connections and disconnections with a connector insertion loss ≦1 dB—independent of the fiber optic probe character. Total insertion loss of ≦5 dB is maintained, and includes consistent alignment of the polarization plane, the connector, the splice at the proximal polarizer, the polarizer, and the splice at the distal polarizer. Total polarization quality of the emitted light is maintained throughout normal use of the fiber optic probe in and out of an endoscope channel with expected bending and vibration when used by a physician. A polarization extinction ratio of ≦−30 dB can also be achieved. In addition the proximal portion of the proximal end of the fiber optic probe incorporates strain relief (with additional vibration dampening capability), the polarizer, connectors, and integration with the PEEK tubing on the probe.

A disposable cover for a fiber optic probe is shown in FIG. 12 and generally designated at 200. The disposable cover comprises a self-tensioning device on a proximal end of the cover and an optical window on a distal end of the cover that provides for alignment with the optical elements of the distal end of the fiber optic probe. The disposable cover may be coupled to, or engaged by, the proximal end connector described herein in order to form a complete unit for endoscopic application of a fiber optic probe.

The disposable cover 200 provides a protective barrier between the fiber optic probe assembly and the patient being endoscopically examined. The disposable cover 200 allows the fiber optic probe assembly to be quickly used again in another patient without high level decontamination or disinfection. The fiber optic probe enters the disposable cover 200 through a proximal opening 210 in the handle 201 (FIG. 16). During insertion the distal probe tip 101 of the fiber optic probe assembly proceeds through a spring guide 202, a spring 203, a retaining ring 204, a strain relief 205, a sheath 206, and a distal housing 207. The distal tip of the fiber optic probe is inserted till it comes into physical contact with the proximal surface 212 of an optical window 208 that is held in the distal housing by optical adhesive 209 (FIG. 18). Once the probe is fully inserted the handle is pulled back till the locking feature 211 shown in FIG. 16 can be locked on the mating pins 105 of the proximal end connector shown in FIG. 4. The action of locking the disposable cover onto the proximal end connector compresses the spring and creates tension in the sheath. This tension insures contact will be maintained between the distal probe tip of the fiber optic probe assembly and the optical window of the disposable cover during use.

The disposable cover as shown in FIG. 12 comprises a handle 201 for structural integrity of the proximal end, a spring guide 202 for capturing and directing the spring, a spring 203 for providing tension into the sheath when coupled or engaged with the proximal end connector 100, a retaining ring 204 for holding the spring guide in the handle against the force of the spring, a strain relief 205 for preventing the sheath and probe from kinking near the exit of the spring guide, a sheath 206 shown with break lines for providing a flexible barrier between the fiber optic probe assembly and the patient, a distal housing 207 for providing a housing for the optical window and alignment of the probe tip, an optical window 208 for providing a fixed barrier between the fiber optic probe assembly and patient that allows emitted light to be transmitted from the probe tip to the tissue under examination and the remitted, scattered light to return to the fiber optic probe assembly, and an optical adhesive 209 for holding the optical window in the distal housing.

As an alternative to using a retaining ring to hold the spring guide in the handle, the embodiment of FIG. 14 describes a handle 220 for the disposable cover that comprises proximal 221 and distal 222 segments, threaded 223 and locked together using a self-locking mechanism. The smaller inner diameter of the proximal segment relative to the distal segment provides a bearing surface for holding the spring guide 202 in the handle against the force of the spring 203. The smaller inner diameter of the proximal segment relative to the spring guide provides smooth insertion of the probe through the handle and spring guide into the sheath 206 of the disposable cover. FIG. 15 illustrates the assembled disposable cover showing the proximal and distal segments threaded to together and locked, along with the spring guide attached to the strain relief.

Referring to FIGS. 12 and 16, the disposable cover visible components comprise a handle 201, a spring guide 202, a retaining ring 204, a strain relief 205, a sheath 206, and a distal housing 207. The probe enters the disposable cover through the proximal opening 210 in the handle 201. Once the fiber optic probe is fully inserted into the disposable cover the handle is pulled back till the locking feature 211 can be locked on the pins 105 of the proximal end connector.

Referring to FIGS. 16 and 18, the distal end of the disposable cover 200 contains a sheath 206, a distal housing 207, an optical window 208, and optical adhesive 209. The proximal surface 212 of the optical window 208 interfaces with the distal surface of the probe and the distal surface 213 of the optical window 208 interfaces with the patient. Various methods can be used to bond the sheath to the distal housing such as a mechanical bond using barbs, using a sheath material that could be heat shrunk onto the housing, or using an adhesive which would require a gap 214 for the adhesive to form the bond. Further embodiments for the components of the distal end are set forth in FIGS. 23 A-I and the accompanying figure legends.

The proximal end of the disposable cover 200 contains a handle 201, a spring guide 202, a spring 203, a retaining ring 204, a strain relief 205, and a sheath 206. The spring 203 is initially compressed between the handle and the spring guide a set distance 215 that is controlled by the retaining ring not allowing the spring to force the spring guide into the handle. The retaining ring is set by the groove 216 that is physically cut into the spring guide. Another embodiment would be to have the strain relief also act as the retaining ring. Various methods can be used to bond the sheath to the spring guide such as a mechanical bond using barbs, a compression nut, or using an adhesive which would require a gap 217 for the adhesive to form the bond. Various methods can be used to bond the strain relief to the spring guide such as a mechanical bond using barbs, using a strain relief material that could be heat shrunk onto the housing, or using an adhesive which would require a gap 218 for the adhesive to form the bond.

Various types of springs could be used. The spring in one embodiment is a compression spring with closed and ground ends, but others such as a wave spring, a coil spring, or a set of Belleville washers could be used also.

A tensioner at the proximal end of the disposable cover as described above has two advantages. It first limits the force that can be applied onto the distal tip optical window of a disposable cover by pulling on the handle. The second advantage is it can absorb any stretching of the sheath and still maintain contact between the probe distal tip and the proximal surface of the optical window while also preventing bunching or pinching of the sheath material through multiple insertions into an endoscope channel.

Once the probe 100 is fully inserted into the disposable cover 200 with the distal tip of the probe 101 in physical contact with the proximal surface 212 of an optical window 208, the handle 201 is rotated to align the locking feature 211 with the mating pins 105.

Referring to FIGS. 21 and 22, once the disposable cover 200 is aligned to the probe 100, the handle 201 is pulled back till the locking feature 211 can be locked by rotation 219 on the mating pins 105. The action of locking the disposable cover onto the probe compresses the spring 203 not shown and creates tension in the sheath 206. This tension insures contact will be maintained between the probe tip at the distal end of the fiber optic probe and the optical window of the distal end of the probe cover during use. The tension from the spring provides the force to keep the pins 105 from backing out of the locking feature.

The preferred sheath material is selected to have a low friction coefficient to allow insertion of the fiber optic probe and insertion into the endoscope channel. Preferably, the material will not plastically deform under tension to the point that the spring is fully released and thus tension is lost and the optical window will no longer contact the fiber optic probe. The sheath material is preferably strong enough to not lose the microbial barrier function by ripping or puncturing from multiple uses with a patient. It must be able to bond to the spring guide and distal housing with enough force that those bonds will not break under high loads. High-density polyethylene meets all of the requirements. Another embodiment would be to use polytetrafluoroethylene which would allow the disposable cover to be peelable for removal.

The correct optical window material must be selected to allow enough light to be transmitted through the window and maintain the polarization axis without loss of the light from the plane of polarization. The preferred material is glass for transmitting and maintaining polarization, but it could be optically transparent, non-polarizing plastic for improved cost and manufacturability.

In another embodiment, the disposable cover is to have the spring guide, spring, and handle as part of the probe assembly and to integrate into the handle a locking mechanism for attachment and removal of a funnel on the disposable cover. This design has the advantage of less parts and thus lower cost on the disposable cover with more parts and higher cost on the reusable probe.

The disposable cover 200 may be packaged into a tray that will protect the sheath and allow insertion of the fiber optic probe while the disposable cover remains protected in the packaging. The disposable cover 200 can be sterilized either alone or in combination with its packaging. When complete, the disposable cover 200 will be contained in a thermoformed tray with Tyvek lidding as the sterile barrier. Such an arrangement would provide a single level packaging and a quantity of these would be put in a box to form a sales unit package level.

FIG. 24 illustrates one embodiment of a complete low coherence interferometry system. The system comprises a component housing an optical engine for fa/LCI 300, a fiber probe assembly comprising a proximal end connector 100 configured to allow replacement or interchange of a fiber optic probe, a fiber optic bundle, PM delivery fiber, distal tip with optical element, and a disposable cover 200 comprising a proximal end further comprising a self-tensioning handle capable of being coupled to the proximal end connector of the fiber probe assembly and a distal end comprising an optical window that aligns and associates with the optical element aligned and associated with a fiber optic probe comprising a fiber optic bundle and a PM fiber. The entire fiber optic probe assembly with coupled disposable cover is shown attached to the optical port on the component housing the optical engine. The system further comprises a medical grade, touch-screen with integrated computer processing unit and memory storage 500 on a wheeled cart 400 for ease of movement and transport in an endoscopic suite.

Although embodiments disclosed herein have been illustrated and described herein with reference to preferred embodiments and specific examples thereof, it will be readily apparent to those of ordinary skill in the art that other embodiments and examples can perform similar functions and/or achieve like results. Various modifications to the disclosure will be readily apparent to those skilled in the art, and the generic principles defined herein may be applied to other variations without departing from the spirit or scope of the disclosure. All such equivalent embodiments and examples are within the spirit and scope of the present invention and are intended to be covered by the appended claims.

It will also be apparent to those skilled in the art that various modifications and variations can be made to the present invention without departing from the spirit and scope of the invention. Thus, the disclosure is not intended to be limited to the examples and designs described herein, but is to be accorded the widest scope consistent with the principles and novel features disclosed herein. For example, the present invention is not limited to a particular Fourier domain or angle-resolved optical biopsy system, tissue type examined, therapy or therapeutic, an endoscope or endoscopic probe, control systems or interfaces, or methods, processes, techniques disclosed herein and their order.

The embodiments set forth above represent the necessary information to enable those skilled in the art to practice the invention and illustrate the best mode of practicing the invention. Upon reading the following description in light of the accompanying drawings figures, those skilled in the art will understand the concepts of the invention and will recognize applications of these concepts not particularly addressed herein. It should be understood that these concepts and applications fall within the scope of the disclosure and the claims that follow.

We claim:

1. A fiber optic probe assembly, comprising:
an optical engine;
a fiber optic probe having a proximal end and a distal end, the fiber optic probe including a plurality of fibers extending between the proximal end and distal end of the fiber optic probe; and
a proximal end connector, comprising
a self-aligning connector for connecting the optical fibers of the fiber optic probe to the optical engine; and
an integrated fiber optic-based polarizer.

2. The fiber optic probe assembly of claim 1 further comprising an embedded memory.

3. The fiber optic probe assembly of claim 2 further comprising an RFID tag.

4. The fiber optic probe assembly of claim 2 wherein the embedded memory comprises
a unique identifier for the fiber optic probe assembly;
a sensor for detecting connection and disconnection of the fiber optic probe to the optical engine; and
an event counter for recording connection and disconnection events.

5. A disposable cover for enclosing at least a portion of a fiber optic probe assembly including a fiber optic probe having a projection at proximal end and optical elements at a distal end, the disposable cover comprising:
an elongated tubular housing having a proximal end and a distal end;
a sheath portion adapted to receive at least a portion of the fiber optic probe between the ends of the housing;
a handle disposed on the proximal end of the housing, the handle comprising a self-tensioning device; and
an optical window disposed on the distal end of the housing, the optical window adapted to provide for alignment with the optical elements of the fiber optic probe.

6. The disposable cover of claim 5 wherein the self-tensioning device comprises a spring disposed inside the handle such that when the handle is enclosing at least a portion of the fiber optic probe assembly the sheath is held under tension.

7. The disposable cover of claim 5 wherein the handle defines a locking slot having a longitudinal portion extending from a proximal end of the handle and opening into a partial peripheral portion, the slot adapted to receive the projection at the proximal end of the fiber optic probe for locking the position of the handle relative to the fiber optic probe.

8. A low coherence interferometry system, comprising:
a fa/LCI device;
a fiber probe assembly, including a replaceable fiber optic probe having optical elements at a distal end; and
a disposable cover for enclosing at least a portion of the fiber optic probe assembly, the disposable cover comprising
an elongated tubular housing having a proximal end and a distal end, the housing including a sheath portion for receiving at least a portion of the fiber optic probe between the ends of the housing;
a handle disposed on the proximal end of the housing, the handle comprising a self-tensioning device; and
an optical window disposed on the distal end of the housing, the optical window providing for alignment with the optical elements of the fiber optic probe.

9. The low coherence interferometry system of claim 8, wherein the fiber optic probe assembly further comprises an embedded memory.

10. The low coherence interferometry system of claim 9, wherein the fiber optic probe assembly further comprises an RFID tag.

11. The low coherence interferometry system of claim 9, wherein the embedded memory comprises
a unique identifier for the fiber optic probe assembly;
a sensor for detecting connection and disconnection of the fiber optic probe to the optical engine; and
an event counter for recording connection and disconnection events.

12. The low coherence interferometry system of claim 7, wherein the self-tensioning device comprises a spring disposed inside the handle such that when the handle is enclosing at least a portion of the fiber optic probe assembly the sheath is held under tension.

13. The low coherence interferometry system of claim 9 wherein the handle defines a locking slot having a longitudinal portion extending from a proximal end of the handle and opening into a partial peripheral portion, the slot configured for receiving a projection at a proximal end of the fiber optic probe.
